(19) United States
(12) Patent Application Publication
(10) Pub. No.: US 2018/0133353 A1
(43) Pub. Date: May 17, 2018

(54) SCENT CONTROL COMPOSITIONS

(71) Applicants: Carlos Diego Garcia, San Antonio, TX (US); George R. Negrete, San Antonio, TX (US)

(72) Inventors: Carlos Diego Garcia, San Antonio, TX (US); George R. Negrete, San Antonio, TX (US)

(73) Assignee: THE BOARD OF REGENTS OF THE UNIVERSITY OF TEXAS SYSTEM, Austin, TX (US)

(21) Appl. No.: 15/574,395

(22) PCT Filed: May 16, 2016
(86) PCT No.: PCT/US16/32636

§ 371 Date: Nov. 15, 2017

Related U.S. Application Data
(60) Provisional application No. 62/162,107, filed on May 15, 2015.

(51) Int. Cl. A61L 9/012; A61L 9/14

(52) U.S. Cl. CPC A61L 9/012; A61L 9/14

(57) ABSTRACT

Certain embodiments are directed to liposome compositions comprising a synthetic lipid composition encapsulating a scent suppressing solution.

### CROSS-REFERENCE TO RELATED APPLICATIONS

This application claim the benefit of priority of U.S. provisional patent application No. 62/162,107, filed May 15, 2015, which is hereby incorporated by reference in its entirety.

### BACKGROUND

Certain embodiments relate generally to deodorizing and scent eliminating compositions.

Deer and other wild game use their senses of smell, sight, and hearing to detect and avoid their natural enemies. For most large game animals, their sense of smell is their greatest defense. Deer and other trophy animals typically travel into the wind and rely on their sense of smell to warn them of danger. Big trophy animals will avoid an area when they detect the presence of a human, or even when they detect that a human has been there. What warns them is primarily human scent from a hunter being present and residual human scent on anything touched by the hunter's hands, clothing, boots, and equipment. In addition, wild game can smell and avoid unnatural scents from weapons, tree stands, backpacks, and other hunting equipment and accessories. These human and equipment scents tend to settle and pool, and then they are spread by the wind in the hunting area generally and particularly downwind of the hunters.

Hunters often use deodorants and scent eliminating sprays to avoid scent detection. Some scent eliminators merely cover or mask a hunter's scent, which allows prey to still detect a scent. A common ingredient found in these scent eliminators is sodium bicarbonate or baking soda, which can disadvantageously leave a white powdery residue on the user's clothing when applied. Other prior art scent eliminators utilize carbon as a base ingredient, which can disadvantageously stain clothing or bleed or fade clothing colors when is applied. In addition many of the currently available scent eliminators fight or suppress odors for only a short period of time.

There remains a need for additional scent eliminating compositions.

### SUMMARY

Certain embodiments are directed to lipidic particle or liposome compositions comprising an amino acid lipid analog encapsulating a scent suppressing solution. In certain aspects cholesterol, cholesterol analogs, and linear fatty acids also can be used as liposome components. In certain aspects cholesterol and its analogs, as well as linear fatty acids are used in combination with amino acid lipid analogs. In certain aspects the scent suppressing solution comprises an oxidant, such as ozone or peroxides (e.g., hydrogen peroxide), for example a peroxide/bicarbonate solution. A peroxide is a compound containing an oxygen-oxygen single bond or the peroxide anion ([O—O]²⁻). The O—O group is the peroxide group or peroxo group. The simplest stable peroxide is hydrogen peroxide, H₂O₂. In certain aspects the liposomes are multilamellar liposomes (MLVs) comprising a lipid and amino acid lipid analog.

An amino acid lipid analog can be selected from an asparagine, serine, or cysteine amino acid lipid analog. In certain aspect amino acid lipid analog can have hydrocarbons of 9, 10, 11, 12, 13, 14, 15, 16, 17, 18, 19, or 20 carbons long. In certain aspects the amino acid lipid analog is an asparagine lipid analog (ALA). In a further aspect the ALA has 11 and 17 carbon hydrocarbons (ALA_{11,17}).

Ratio of lipid to amino acid lipid analog can be varied. In certain aspects the ratio of lipid to amino acid lipid analog is about 5, 6, 7, 8, 9, 10, 11, 12, 13, 14, 15, or 16 to 0.5, 1, or 2. In a further aspect a lipid to amino acid lipid analog ratio is about 9:1. Certain embodiments are directed to a lipid particle or liposome composition comprising: at least 5, 10, 15, 20, 40, or 60 mol %, including all values and ranges there between, of an amino acid lipid analog; at least 40, 60, 80, 90, 95 mol % amphiphilic lipid; and a scent suppressing solution. In certain aspects the compositions comprise 0.1, 0.5, 1, 5, 10, or 20 mol % of cholesterol, cholesterol analog, or linear fatty acids, including all values and ranges there between.

As used herein, “analog” refers to a chemical compound that is structurally similar to a parent compound, but differs in composition (e.g., differs by appended functional groups or substitutions). Analog as used here refers to a compound having chemical or physical properties similar to the original compound.

The scent suppressing solution can comprise an oxidant such as ozone or peroxide at 2, 3, 4, 5 or 6% (w/w). The solution can be buffered using buffers such as bicarbonate, phosphate, citrate, and acetate buffers. The solution can also comprise a salt such as sodium chloride.

Certain embodiments are directed to a container configured to spray or aerosolize a scent suppressing composition described herein. In certain aspects the composition is formulated as a liquid, gel, lotion, or cream that can be dispersed on the surface of a subject or object to reduce the scent of the subject or object.

Certain embodiments employ the liposomal compositions as a deodorant and/or scent suppressor. Other embodiments can use the liposomal composition loaded with an odiferous compound or composition and provide for the controlled release of such compounds.

Methods of preparing liposome composition can comprise hydrating a thin film and cycling the hydrated thin film between freezing and thawing. In certain aspects the composition is frozen and then heated to a temperature that is at the phase transition of the liposome composition. The liposome compositions can be subjected to 3, 4, 5, 6, 7 or more cycles of freeze/thaw.

Certain embodiments are directed to methods of reducing the scent of a subject and/or object by contacting the subject and/or object an effective amount of a liposome composition described herein. As used herein, an “effective amount” refers to the quantity of a liposome composition required to reduce the amount of odiferous molecules emanating from a subject and/or object. In certain aspects compositions are sprayed, rubbed, and/or dispersed on the clothes, skin, or surface of an individual and/or object.

Other embodiments of the invention are discussed throughout this application. Any embodiment discussed with respect to one aspect applies to other aspects as well and vice versa. Each embodiment described herein is understood to be an embodiment that is applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects. Embodiments may be better understood by reference to one or more of these drawings in combination with the detailed description of the specification.

FIGS. 1A-1B. (FIG. 1A) Gas Chromatography spectrum of the sweat extracted from water (liquid extraction), showing squalene at 15.83 min. (FIG. 1B) Mass spectrum for retention time=15.983 min. showing squalene at relative abundance.

FIGS. 2A-2B. (FIG. 2A) Gas Chromatography spectrum of the sweat extracted from water (liquid extraction) with Scent Killer added, showing squalene at 15.83 min. (FIG. 2B) Mass spectrum for retention time=15.983 min. showing squalene at high abundance.

FIGS. 3A-3B. (FIG. 3A) Gas Chromatography spectrum of the sweat extracted from water (liquid extraction) with Evolve 3D added, showing squalene at 15.83 min. (FIG. 3B) Mass spectrum for retention time=15.983 min. showing squalene at high abundance (in large quantity).

FIGS. 4A-4B. (FIG. 4A) Gas Chromatography spectrum of the sweat extracted from water (liquid extraction) with Bone Collector added, showing squalene at 15.83 min. (FIG. 4B) Mass spectrum for retention time=15.983 min. showing squalene at high abundance.

FIGS. 5A-5B. (FIG. 5A) Gas Chromatography spectrum of the sweat extracted from water (liquid extraction) with EliminX added, showing squalene at 15.83 min. (FIG. 5B) Mass spectrum for retention time=15.983 min. showing squalene at high abundance.

FIGS. 6A-6B. (FIG. 6A) Gas Chromatography spectrum of the sweat extracted from water (liquid extraction) with liposomal solution added, showing squalene at 15.83 min. (FIG. 6B) Mass spectrum for retention time=15.983 min. showing squalene at low abundance.

FIG. 7. Graph comparing the efficiency of four commercial sprays against the formulation developed at UTSA.

FIG. 8. Gas chromatogram obtained with the headspace of used cat litter.

FIG. 9. Gas chromatogram obtained with the headspace of used cat litter after treatment with the spray containing liposomes.

FIG. 10. Structure and names of the most abundant compounds found in the headspace of used cat litter.

### DESCRIPTION

A number of commercial hunting sprays offer “high” levels of short-term scent reduction/elimination. The components of these sprays are typically not disclosed. These sprays are used for extended times in the “field” where temperature extremes, rough terrain and obstacles requiring physical stamina and performance may cause a great amount of perspiration. This perspiration contains bodily metabolites that are volatile or fuel bacteria on the skin to release small, odiferous molecules. The effective duration of these hunting sprays is not ideal for extended periods of time.

Certain embodiments of the compositions described herein provide a longer lived scent suppressing composition. As one example, a peroxide:bicarbonate solution was encapsulated within liposomes comprised of distearoylphosphatidylcholine (DSPC) and a synthetic lipid asparagine-lipid analog (ALA). Application of such a composition provides short-term scent prevention by disinfecting the area of application and/or long term-term scent protection due to deposition of liposomes that are permeable to volatile organic compounds.

An example of a liposome composition described here was tested for scent elimination efficiency against four commercially available hunting spray-deodorants. Samples of human sweat were collected and analyzed via gas chromatography coupled mass spectrometry (GC-MS). Although many volatile, organic molecules are normally present in “body odor,” squalene was selected as a proxy for scent to quantify the efficiency of the proposed spray. Squalene is a fatty triterpene secreted in sebum that acts as a scent precursor when consumed by bacteria. Studies show that a multilamellar vesicles (MLV) solution results in a greater reduction in squalene than the four commercial sprays. The liposome compositions provide the capture and oxidation of human scent and also provide a long-term method to avoid detection by animals.

The studies reported herein used four commercially available scent eliminators to investigated for their efficiency at destroying squalene, one of the molecules found in human odor samples. Liposomal solutions as described herein proved to be the most efficient with a 93.5% reduction of squalene. MLVs were characterized by scanning electron microscopy to visualize the nanocapsules.

### I. LIPOSOME COMPOSITIONS

Liposomes are nano- to micro-scale lamellar vesicles capable of encapsulating hydrophobic cargo within a bilayer or water-soluble cargo in an aqueous interior. Liposomes have become increasingly important as a method of delivery for drugs, nutrients, cosmetic agents, and other chemotherapeutic agents. (Torchilin, Nat Rev Drug Discov 2005, 4(2): 145-60; Gabizon, Liposomal drug carriers in cancer therapy. In Nanoparticulates as Drug Carriers, Torchilin, V. P., Ed. Imperial College Press: London, United Kingdom, 2006; pp 437-62; Stanzi, Cosmetic Science and Technology. 2nd ed.; Marcel Dekker, Inc.: New York, N.Y., 1999; Vol. 19; Lasic and Barenholz, Handbook of Nonmedical Applications of Liposomes. CRC Press, Inc.: Boca Raton, Fl., 1996). Liposomes comprise a lipid component that forms the bilayer. Lipids constitute a group of naturally occurring molecules that include phospholipids. One of the main biological functions of lipids is as structural components of cell membranes. Lipids forming liposomes are amphiphilic small molecules with the amphiphilic nature allowing them to form structures such as vesicles, liposomes, or membranes in an aqueous environment.

Liposomal lamellar vesicles can be characterized into three categories: small unilamellar vesicles (SUVs), which consist of a single bilayer and range in diameter from 50-100 nm; large unilamellar vesicles (LUVs), which consist of a single bilayer and range in diameter from 100-250 nm; or multilamellar vesicles (MLVs), which are large vesicles consisting of concentric spheres of lipid bilayers with regions of aqueous media between them, ranging in diameter from 100-1000 nm. MLVs can be sonicated to reduce the size of the nanocapsules, as well as to decrease the degree of lamellarity.

Generally, liposomes are assembled from phosphatidylcholine-based lipids including distearoylphosphatidylcholine (DSPC). Liposome stability can be enhanced by altering the composition or including cholesterol or other stabilizing compounds in the formulation. Synthetic lipids have also been included in formulations for enhanced stability (Mfuh et al. Langmuir 27(8):4447-55). Liposomes have been used to encapsulate various biologically relevant compounds including ⁹⁹Tc- and ¹⁸⁶Re-labeled doxorubicin (Head et al., Radiology 255(2):405-14; Soundararajan et al., Nuclear Medicine and Biology 2009, 36(5):515-24) and other powerful chemotherapeutic agents (Muppidi et al., Antimicrob Agents Chemother 55(10):4537-42), quantum dots (Papagiannaros et al., Nanomedicine 2009, 5(2):216-24; Wang et al., J Fluoresc 21(4):1635-42) and even enzymes (Chaize et al., Biosensors and Bioelectronics 2004, 20(3):628-32; Gibbons et al., Pharmaceutical Research 2011, 28(9):2233-45; Yoshimoto et al., Enzyme and Microbial Technology 49(2):209-14).

Certain embodiments are directed to the use MLVs to capture human odor. Human odor is caused by the release of volatile organic compounds from metabolism and bacteria on the skin (Curran et al., Journal of Chromatography B 2007, 846(12):86-97; Zhang et al., J Chromatogr B Analyt Technol Biomed Life Sci 2005, 822(1-2):244-52). These volatile compounds can enter the liposome and be acted upon by a scent suppressing solution contained within the liposome.

A “lipidic particle” refers to a particle having a membrane structure in which amphipathic lipid molecules are arranged with their polar groups oriented to an aqueous phase. Examples of the lipid membrane structure include configurations such as a liposome, multi-lamellar vesicle (MLV), and a micelle structure.

A “liposome” refers to a closed nanosphere, which is formed by forming a bilayer membrane of a phospholipid molecule with the hydrophobic moiety positioned inside and the hydrophilic moiety positioned outside, in water and closing the ends of the bilayer membrane. Examples of liposome include a nanosphere having a single layer formed of a phospholipid bilayer membrane and a nanosphere having a multiple layer formed of a plurality of phospholipid bilayers. Since a liposome has such a structure, an aqueous solution is present both inside and outside of the liposome and the lipid bilayer serves as the boundary.

A “micelle” refers to an aggregate of amphipathic molecules. The micelle has a form in which a lipophilic moiety of this amphipathic molecules is positioned toward the center of the micelle and a hydrophilic moiety is positioned toward the outside thereof, in an aqueous medium. A center of a sphere is lipophilic and a peripheral portion is hydrophilic in such a micelle. Examples of a micelle structure include spherical, laminar, columnar, ellipsoidal, microsomal and lamellar structures, and a liquid crystal.

### A. Phospholipids

In addition to distearoylphosphatidylcholine (DSPC) the liposomes may be made from one or more phospholipids including those from natural sources such as plant or animal sources. Phospholipids include but are not limited to phosphatidylcholine, phosphatidylethanolamine, phosphatidylglycerol, sphingomyelin, phosphatidylserine, or phosphatidylinositol. Synthetic phospholipids that may also be used and include, but are not limited to, dimyristoylphosphatidylcholine, dioleoylphosphatidylcholine, dipalmitoylphosphatidylcholine and distearoylphosphatidycholine, and the corresponding synthetic phosphatidylethanolamines and phosphatidylglycerols. Other additives such as cholesterol or other sterols, cholesterol hemisuccinate, glycolipids, cerebrosides, fatty acids, gangliosides, sphingolipids, 1,2-bis(oleoyloxy)-3-(trimethyl ammonio)propane (DOTAP), N-[1-(2,3-dioleoyl)propyl]-N,N,N-trimethylammonium (chloride) (DOTMA), D,L,-2,3-distearoyloxypropyl(dimethyl)-β-hydroxyethyl ammonium (acetate), glucopsychosine, or psychosine can also be added. In certain aspects cholesterol and linear fatty acids can be used in liposome compositions.

The relative amounts of phospholipid, amino acid lipid analogs (see below), and/or additives used in the liposomes may be varied if desired. The preferred ranges are from about 80 to 95 mole percent phospholipid and 5 to 20 mole percent of fatty amino acid derivative, e.g., lipoasparagine, lipocysteine, liposerine; and optionally 5 to 10 mole percent of another additive.

### B. Amino Acid Lipid Analogs

A number of amino acid lipid analogs are described in US Patent Publication 2004/0192894, which is incorporated herein by reference in its entirety. Amino acid lipid analogs comprise a cyclic amino acid conjugated to hydrophobic group(s). Amino acid lipid analogs include, but are not limited to asparagine lipid analogs (ALA), which are also called lipoasparagines; serine lipid analogs (SLA), which are also called liposerines; and cysteine lipid analogs, which are also called lipocysteines. In certain aspects the amino acid lipid analog can have the following general chemical structures:

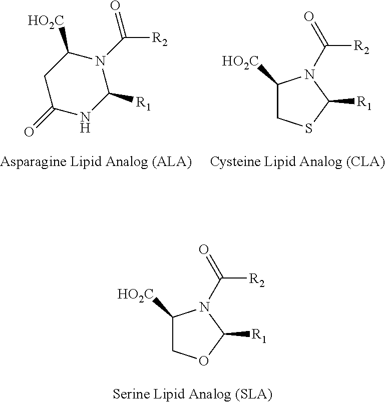

wherein R₁ and R₂ are each, independently, a linear, branched, saturated and/or unsaturated alkyl, a cholesterol moiety, a steroid moiety, an aromatic moiety, a combination thereof. R₁ and R₂ groups can be the same or different. In certain aspects the R₁ and R₂ group is, independently, a saturated or unsaturated 5, 6, 7, 8, 9, 10, 11, 12, 13, 14, 15, 16, 17, 18, 19, 20 or more carbon alkyl group. The alkyl group can be a branched or unbranched alkyl. In certain aspects R₁ is a C9 to C13 alkyl and R₂ is a C15 to C20 alkyl. In a further aspect R₁ is a saturated C11 alkyl and R₂ is a C17 alkyl. In certain aspects the Amino acid lipid analog is an ALA_{11,17} amino acid lipid analog.

The term “alkyl,” by itself or as part of another substituent, means, unless otherwise stated, a linear (i.e. unbranched) or branched carbon chain, which may be fully saturated, mono- or polyunsaturated. An unsaturated alkyl group is one having one or more double bonds or triple bonds. Saturated alkyl groups include those having one or more carbon-carbon double bonds (alkenyl) and those having one or more carbon-carbon triple bonds (alkynyl). The groups, —CH₃ (Me), —CH₂CH₃ (Et), —CH₂CH₂CH₃ (n-Pr), —CH(CH₃)₂ (iso-Pr), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), are all non-limiting examples of alkyl groups.

### C. Scent Suppressing Solutions

In certain aspect the liposome is loaded with a scent suppressing solution. Scent suppressing solutions include scent suppressor such as an oxidant, ozone, hydroxyl radicals, hydroperoxides and combinations thereof. In certain aspects the scent suppressing solution comprises 1, 2, 3, 4, 5, or 6 percent (w/w), including all values and ranges there between, of an oxidant. In certain aspects the oxidant is ozone, peroxides (e.g., hydrogen peroxide) and combinations thereof. The scent suppressor can be in a bicarbonate, phosphate, citrate, or acetate buffered solution, such as sodium bicarbonate at 1, 5, 10, 20, 30, 40, 50, 60, 70, 80, 90, or 100 to 10, 20, 30, 40, 50, 60, 70, 80, 90, or 100 mM. The buffered solution can also comprise a salt such as sodium chloride at 10, 20, 30, 40, 50, 60, 70, 80, 90, 100, or 110 to 100, 110, 120, 130, 140, 150, 160, 170, 180, 190, or 200 mM. In certain aspects the solution is at a pH of 6, 6.5, 7, 7.5, including all values and ranges there between.

### II. EXAMPLES

The following examples as well as the figures are included to demonstrate embodiments of the invention. It should be appreciated by those of skill in the art that the techniques disclosed in the examples or figures represent techniques discovered by the inventors to function well in the practice of the invention, and thus can be considered to constitute modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation and Testing of Scent Suppressing Compositions

Samples of human sweat were collected and analyzed via gas chromatography coupled mass spectrometry (GC-MS). As no small, volatile, odiferous molecules can be realistically considered characteristic of “body odor,” the destruction of scent precursors was monitored using squalene, a fatty triterpene secreted in sebum, as a model compound. Although squalene does not possess a strong odor in low concentrations, it is a precursor to cholesterol and steroids and can be a precursor to odiferous steroids generated by bacteria.

As an example, a scent suppressing compositions comprising DSPC/ALA/peroxide was prepared, tested, and compared to commercially available scent suppressors.

### A. Results

Spray Composition.

Initially, it was thought that the hunting sprays consisted primarily of low concentrations of hydrogen peroxide. To quantify the concentration of peroxide in 4 mL of each hunting spray, a solution of MnO₄₋ was standardized with Na₂C₂O₄. Using the 0.0254M MnO₄₋, each hunting spray was titrated for peroxide. The titrations revealed that two of the commercial sprays (Code Blue EliminX™ “SilverZyme”™ and Evolve 3D™ “Dead Down Wind”™) had 0.003M peroxide as part of the formulation, while the other two (Bone Collector™ “Silver Shield”™ and Scent Killer™ “Super Charged Formula”™) had none.

Liposome Preparation and Visualization.

Liposome compositions were prepared using a freeze-thaw approach resulting in MLVs. Two thin films were prepared and hydrated; one with 25 mL of 10 mM sodium bicarbonate/120 mM sodium chloride and the other with 3% hydrogen peroxide/10 mM sodium bicarbonate/120 mM sodium chloride. Cycling between freezing conditions and heat at the phase transition of the liposome composition, the solutions of ALP_{11,17}/DSPC were subjected to a series of freeze-thawing, forming MLVs.

A 100 μL aliquot of both MLV solutions were stained with a solution 11% (w/w; pH 7.2) of ammonium molybdate ((NH₄)₆Mo₇O₂₄) for visualization by scanning electron microscopy (SEM). A few drops of the stained solution was added to a 300 copper mesh grid, which was fastened to an aluminum mount. Visualization by SEM was poor, so the solution was applied directly to an aluminum mount and sputter-coated with 10 nm Au:Pd. Liposomes prepared without hydrogen peroxide in the hydrating media appeared normal and did not aggregate, while MLVs hydrated with peroxide aggregated into large clumps. Although this aggregation was unexpected, liposomes were present and not compromised.

Gas Chromatography—Mass Spectrometry.

Headspace analysis was performed on the first sample of two 2 inch×2 inch squares sweat containing towel in a control bag. The GC-MS resulted in no molecules of interest, so the sample was pressure-loaded by preventing the sample to exit the sample chamber, concentrating the air and odiferous compounds. Although there was an odor to the cloth squares, no compounds of interest were visible in the analysis, so headspace analysis on the parent towel was performed. Headspace analysis was performed on the bag containing the perspiration-covered towel to see if any organic volatiles were present. Interestingly, none of the normal organic volatiles associated with human sweat/scent were present, so it was decided that extraction of the odiferous compounds present would be the best route. Dichloromethane was used on a 2 inch×2 inch square to extract chemicals that may be causing the scents. As many of the volatile compounds are ketones, aldehydes, alcohols, or acids, and therefore polar, dichloromethane was not expected to extract all of the molecules of interest. GC-MS headspace analysis of the dichloromethane extract did not reveal anything of interest.

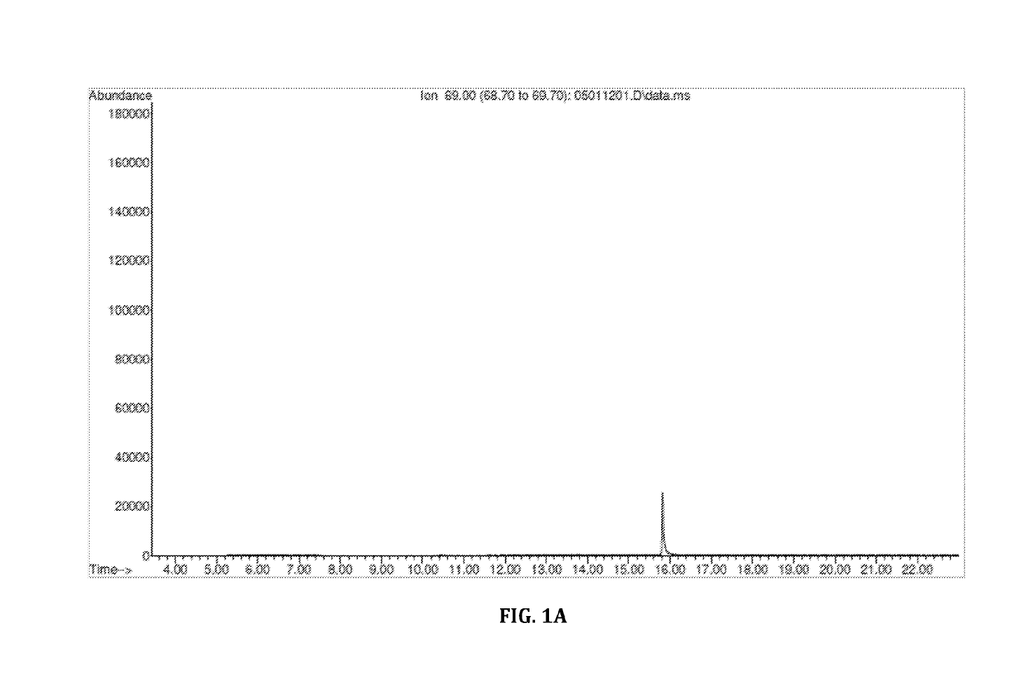

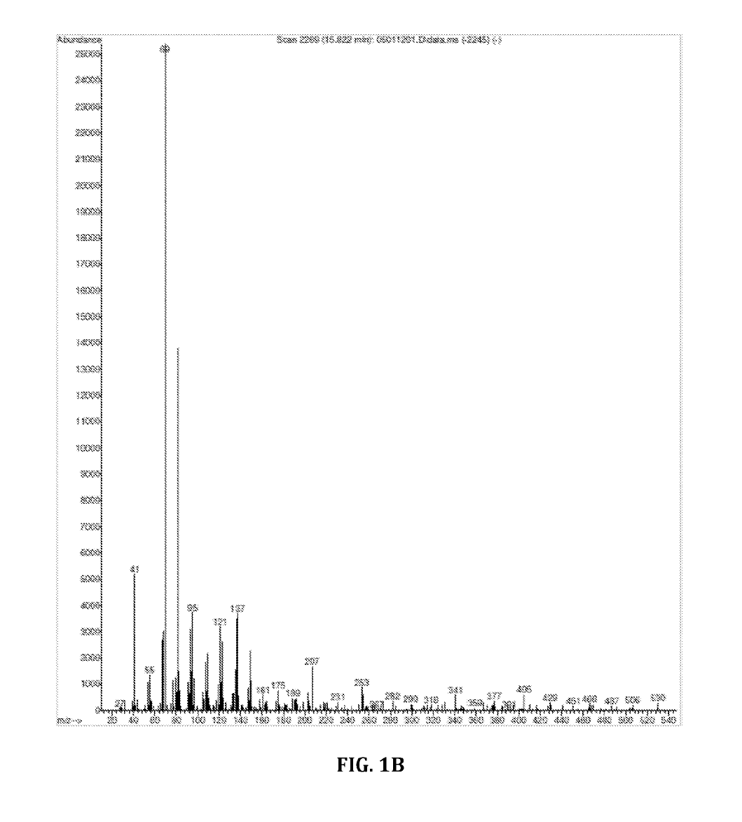

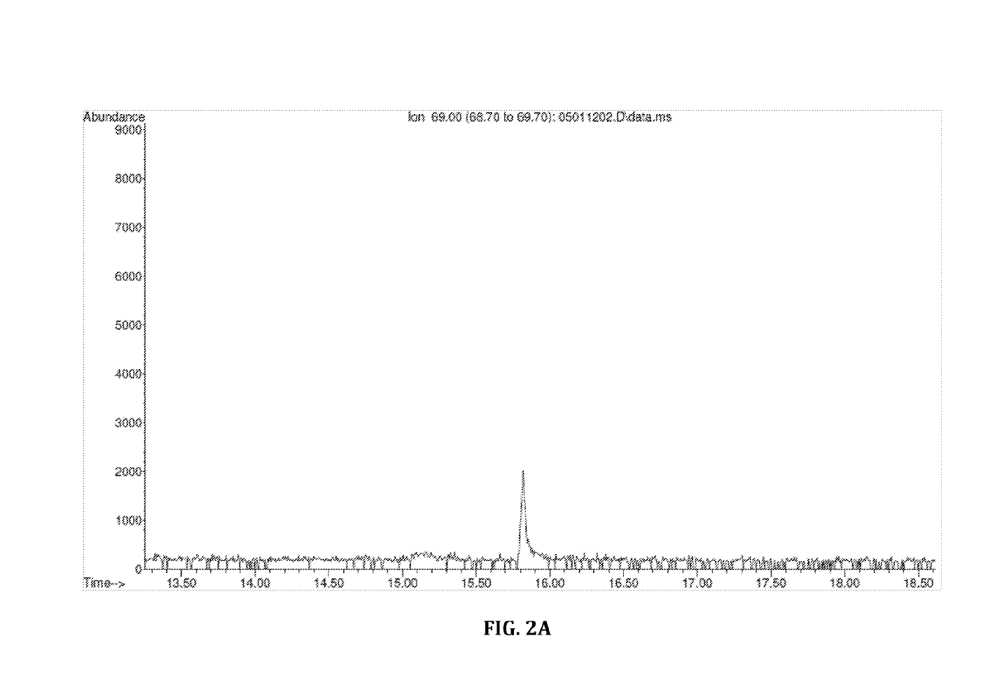

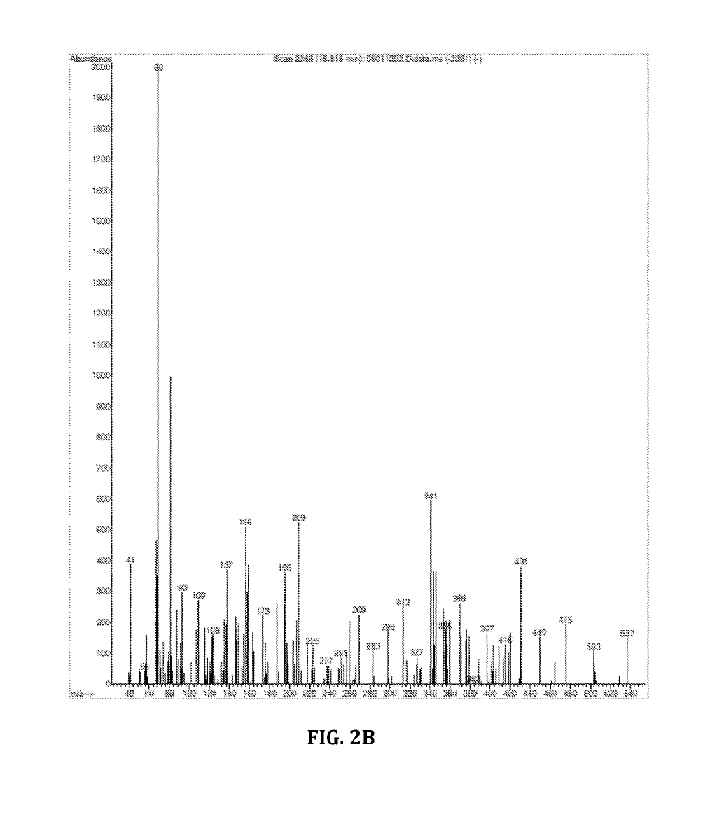

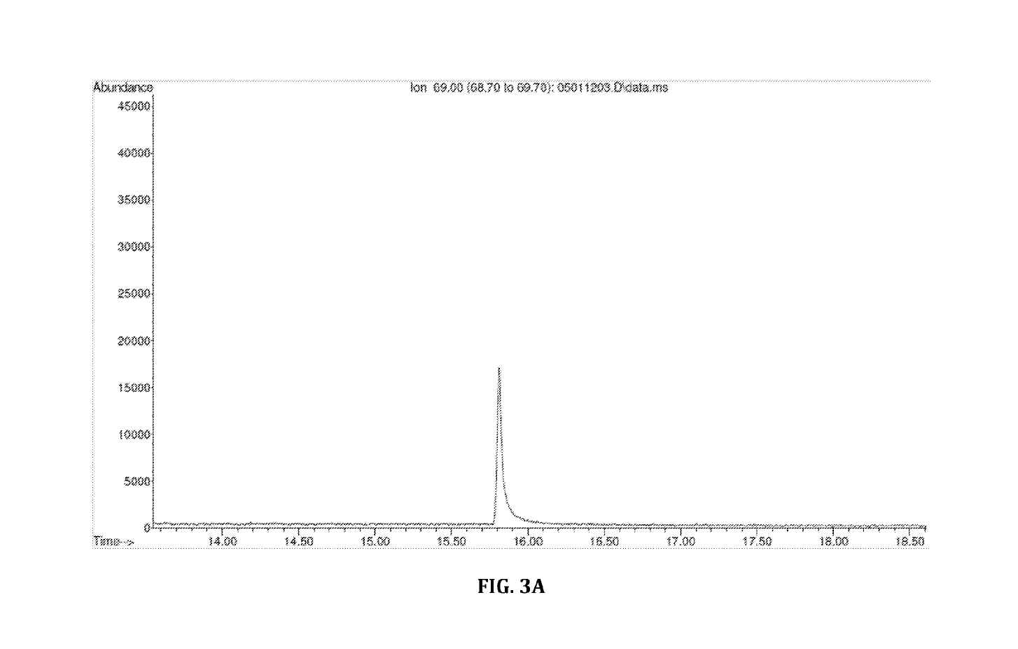

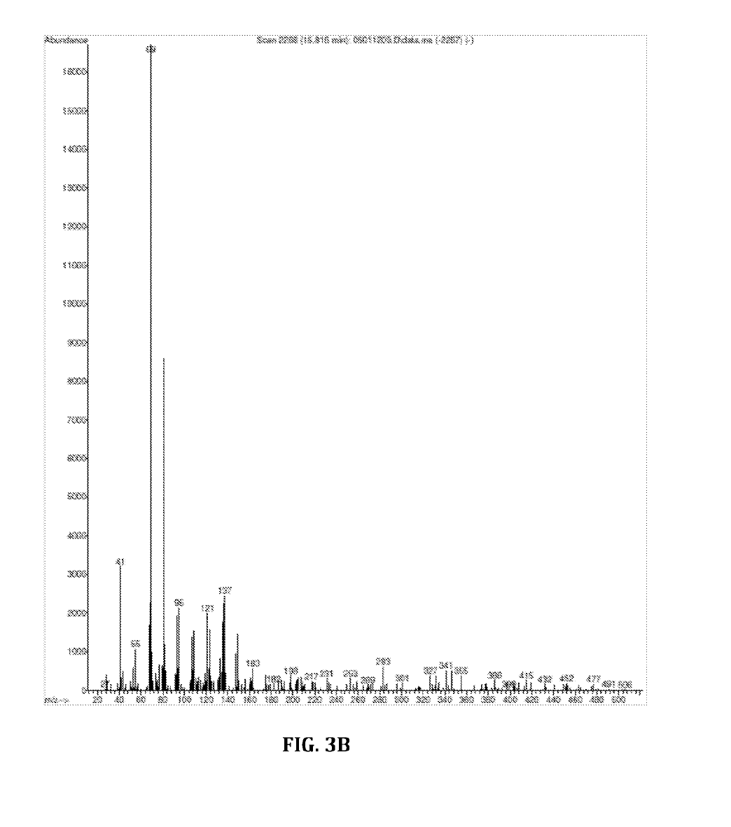

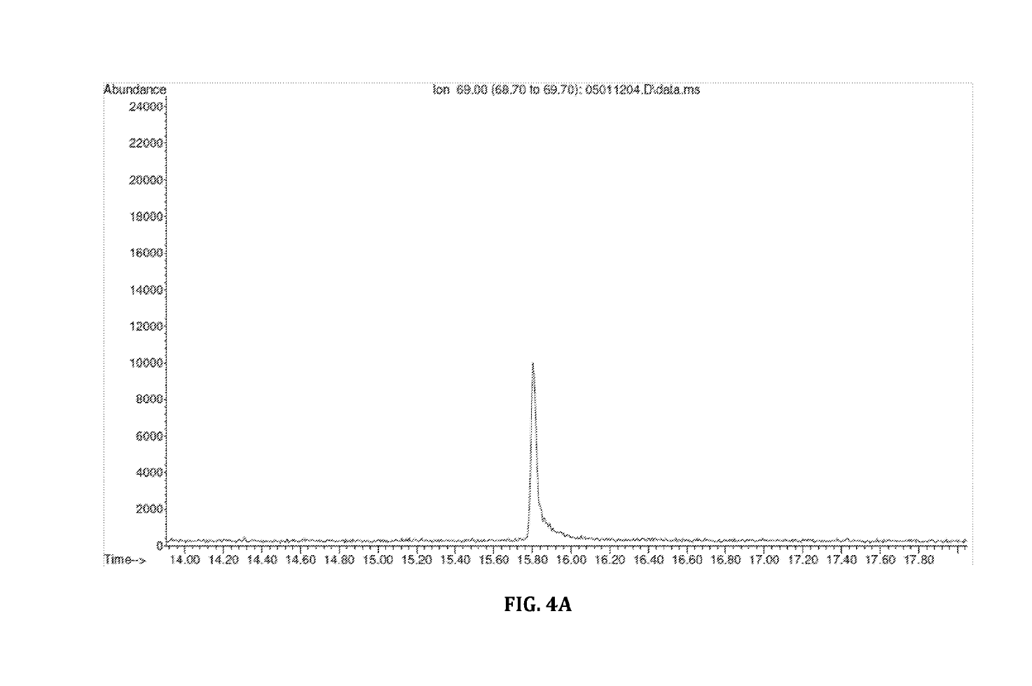

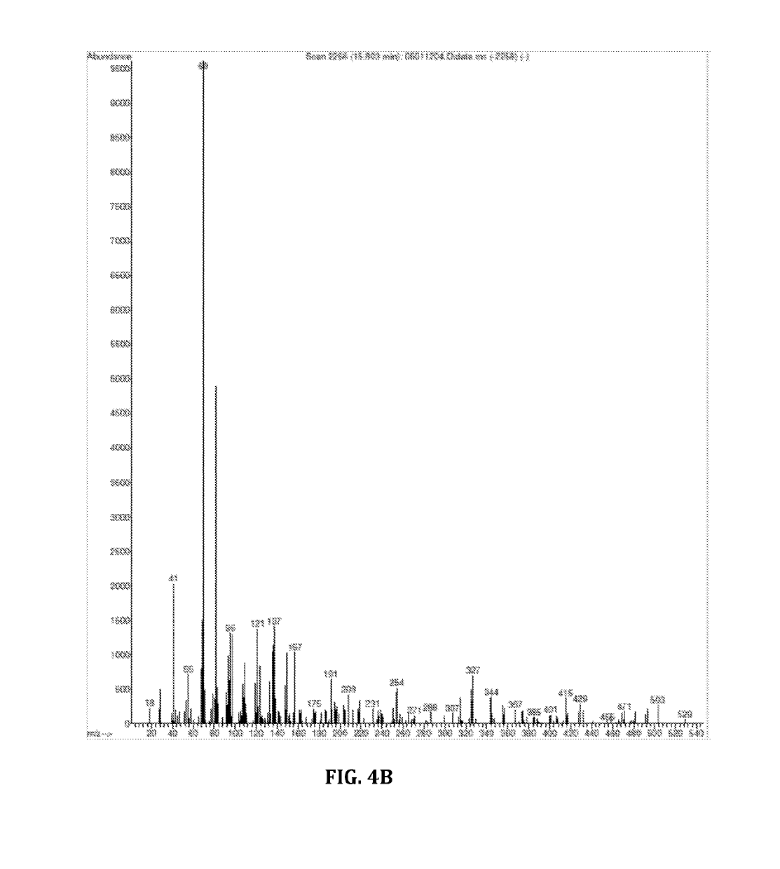

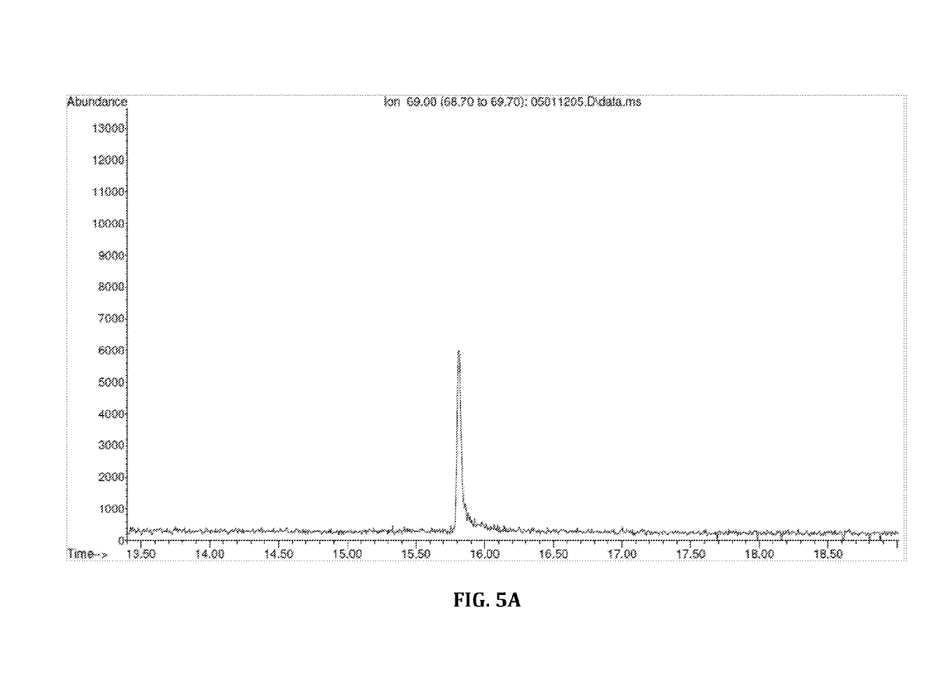

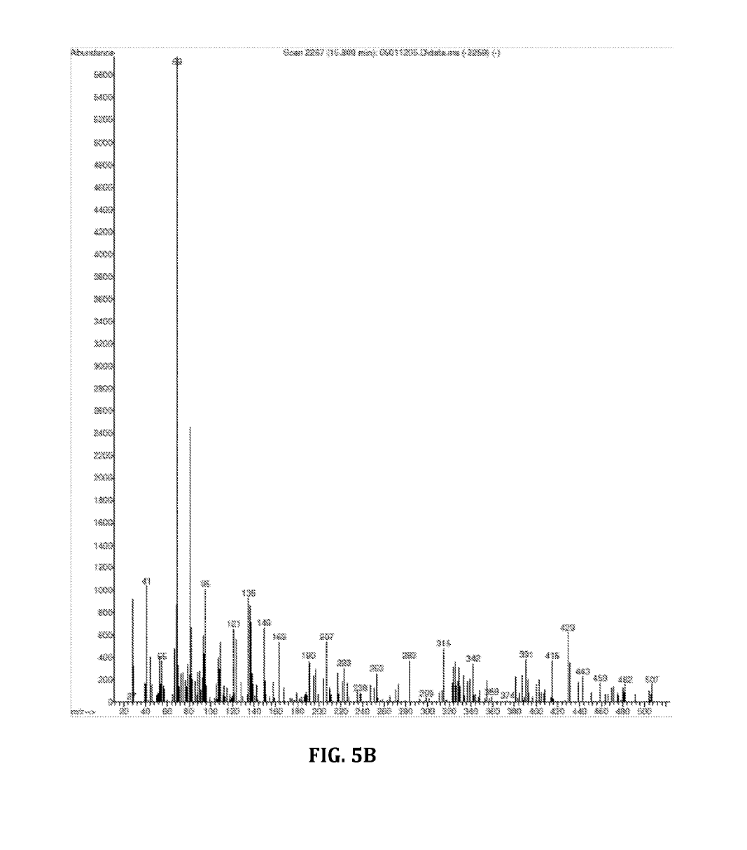

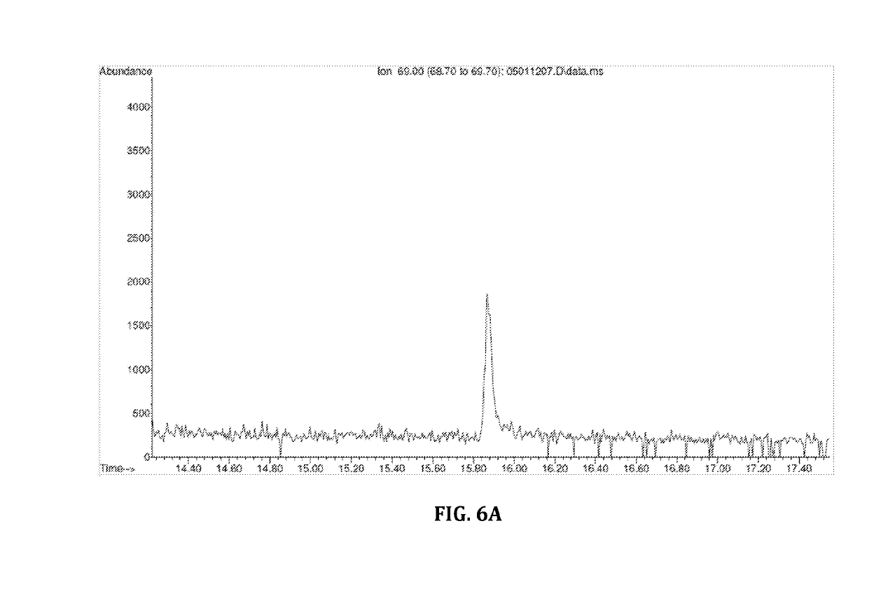

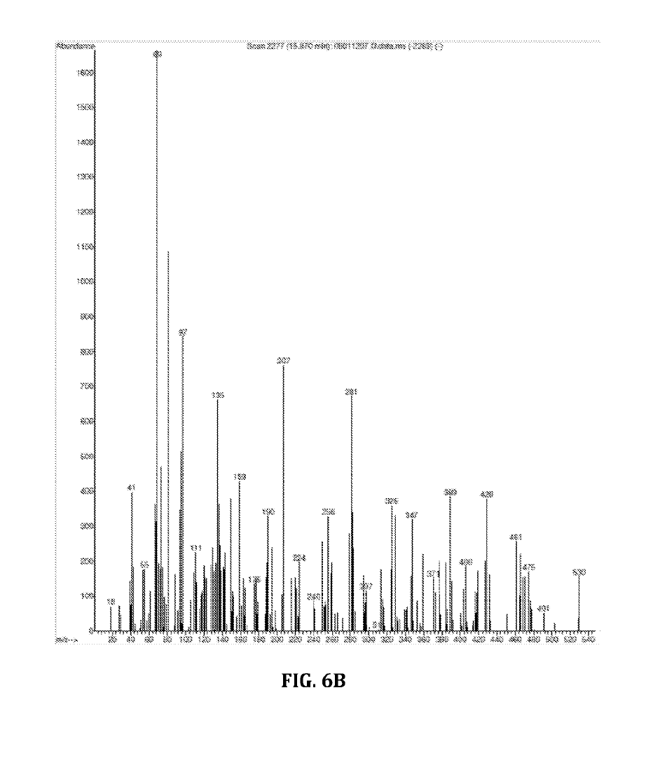

Water was chosen as the next solvent to extract volatiles. Although water broadens peaks on GC, the mass of water is sufficiently low compared to the volatile organics, allowing it to pass the column before the expected organic compounds. The water extraction of sweat revealed squalene, a fatty, unsaturated triterpene secreted in sebum, but is also associated with cholesterol and steroid synthesis within the body. As there is evidence to odiferous steroids produced by bacteria on the skin, squalene was used as a target for oxidation. Squalene was monitored by gas chromatography at retention time of 15.83 minutes, with m/z of 69 (FIGS. 1-6).

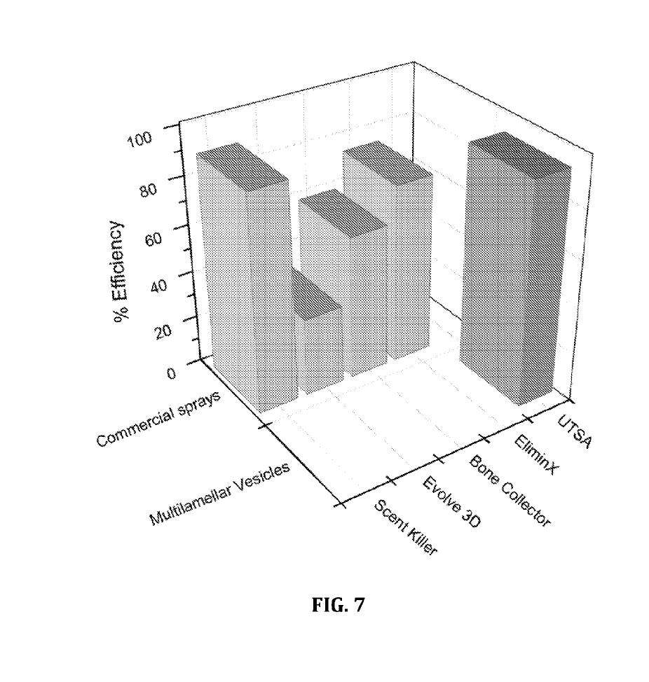

As the hunting sprays are expected to oxidatively disinfect skin and squalene is a candidate for oxidation, the reduction in the amount of squalene per sample was monitored. The results of the reduction of squalene by each scent eliminator (hunting sprays and liposomal solutions) is shown in FIG. 7.

Assuming there was equal distribution of squalene for all samples and that the final volume of samples were all equal, by taking the area for the most intense ion of squalene m/z=69. The area in the sweat extract was used to establish the initial concentration of squalene in each sample; by dividing the area of m/z 69 that was found in each sample at retention time 15.867 min, and multiplying by 100% yielded the % residual amount. Subtracting the residual from 100% gave the overall % Reduction of squalene.

### B. Materials and Reagents

GC/MS was performed using an Agilent 6890N Network GC System with an integrated Agilent 5973 Network Mass Selective Detector. Sonication performed by a Qsonica Q55 probe sonicator. Scanning electron microscopy (SEM) studies were performed on a Jeol JSM-6510LV, using tetramethylsilane (TMS) at δ=0.00 for ¹H. Sodium Oxalate was from Matheson Coleman & Bell. Manganous Sulfate was obtained from Fisher Scientific. KMnO₄ was obtained from J. T. Baker. Distearoylphosphatidylcholine (DSPC) was purchased from Avanti Polar Lipids, Alabaster, Ala. ALA_{11,17} was synthesized and characterized by NMR. NaHCO₃ from Sigma-Aldrich. NaCl from Mallinckrodt. All chemicals were reagent grade.

Four commercial sprays were used as control: Bone Collector™ “Silver Shield”™; Evolve 3D™ “Dead Down Wind”™; Code Blue EliminX™ “SilverZyme”™; Scent Killer™ “Super Charged Formula”™, all purchased from a retail store.

Liposome Preparation.

A mixture of 0.2850 g DSPC and 0.0045 g ALP_{11,17} (9:1 DPSC:ALA) was dissolved in 15 mL chloroform in a round-bottom flask. The solvent was removed through rotary evaporation to acquire a thin film, which was subsequently placed under house vacuum conditions for 1 hour. This thin film was hydrated with 25 mL of either 10 mM sodium bicarbonate: 120 mM sodium chloride or 3% hydrogen peroxide: 10 mM sodium bicarbonate: 120 mM sodium chloride. A probe sonicator was then introduced to the system, and sonication for 5 minutes disrupted the emulsion, forming liposomes.

Multilamellar Vesicle Preparation.

A mixture of 0.2850 g DSPC and 0.0045 g ALA_{11,17} (9:1 DPSC:ALA) was dissolved in 15 mL chloroform in a round-bottom flask. The solvent was removed through rotary evaporation to acquire a thin film, which was subsequently placed under house vacuum conditions for 1 hour. This thin film was hydrated with 25 mL of either 10 mM sodium bicarbonate:120 mM sodium chloride or 3% hydrogen peroxide:10 mM sodium bicarbonate:120 mM sodium chloride. This solution was then cycled between cryogenic conditions (−78° C.) and incubation conditions (58-60° C.) with vortexing to produce MLVs.

MLV Staining for Scanning Electron Microscopy Visualization.

The stability of the liposomes in H₂O₂/HCO₃⁻ media was unknown. 100 μL of liposome or MLV solution acquired through the aforementioned procedures were treated with 25 μL of 11% (w/w; pH 7.2) solution of ammonium molybdate ((NH₄)₆Mo₇O₂₄). This mixture stood exposed to air at room temperature for 12 h. A few drops of the sample was placed on 300 mesh copper grids that were covered in lacy carbon. These grids were allowed to air dry for 2 h. These grids were placed on aluminum mounts for SEM visualization. Images were collected shortly afterwards.

Following the same procedure, samples of the MLV suspension were applied directly to the aluminum mount and coated with ˜10 nm Au:Pd for visualization. Images were collected shortly afterwards.

Gas Chromatography—Mass Spectrometry: Headspace.

2 inch×2 inch squares were cut out of a perspiration-covered towel. Two squares were placed in six plastic quart-sized bags and filled half-full with nitrogen. The bags were allowed to sit for 12 h before a 60 mL syringe was inserted, pumped multiple times, and then a filled a final time for headspace analysis. The syringe sample was transferred immediately to the GC-MS for analysis. After initial headspace experiments, the sample volume was increased by pressure-loading and preventing air samples from leaving the sample chamber of the GC.

Gas Chromatography—Mass Spectrometry: Extraction.

Initial analysis (extraction of sweat): One 2 inch×2 inch square was placed in a vial with 2 mL of dichloromethane and shaken vigorously before being wrung out. The solution was then transferred to the GC-MS instrument for analysis. Full analysis (extraction of sweat; analysis of sprays and liposomal composition; application of sprays and extraction): One 2 inch×2 inch square was placed in a vial with 2 mL of hunting spray. After 5 minutes 2 mL of DI water was added, the vial was shaken vigorously, and then the towel was wrung out. The resulting solution was transferred to the GC-MS for analysis. This was repeated for each hunting spray.

### Example 2

### Preparation and Testing of Scent Suppressing Compositions for Use in Cat Litter

Preparation of Lipids for Thin-Film (Volume of Liposomes 4-25 mL)

Composition: 90% DSPC, 10% ALA (0.000126 total moles lipid per 4 mL)

Weight DPSC per 4 mL: 0.0896 g

Weight ALA_{11,17} per 4 mL: 0.0071 g

Lipids were combined in round-bottom flask and chloroform was added to ensure proper mixing on the components (enough to dissolve all lipids; ˜15-25 mL). After homogenization, the chloroform was extracted in a rotary evaporator, forming a thin film on the glass. It is important to note that the rate of evaporation as well as speed of the rotary evaporation affect the formation of the thin-film. Next, the lipids were placed in a vacuum chamber (minimum 45 minutes) to remove any remaining solvent. Liposomes were then mixed with a solution containing 120 mM NaCl, 3% hydrogen peroxide, and 10 mM bicarbonate. Finally, the solution was subjected to cycles of freezing-storing until the development of a slightly blue hue. Finally, the solution was diluted to the selected concentration and placed in the spray bottles for testing.

Efficiency of the Spray to Neutralize Odor from Cat Urine

In order to determine the efficiency of the spray, new and used cat litter (Tidy Cats) was collected and aliquots placed in zip-lock bags. The headspace of the bags was next analyzed using an Agilent Technologies 6890N GC System (Agilent 122-0132 capillary column) connected to an Agilent 5973 Mass Spectrometer Detector. All samples were analyzed using a flow rate of 0.6 mL/min and a temperature ramp that consisted in the following sequence:
3 min at 45° C.
Ramp to 190° C. for 10 min
Ramp to 240° C. for 20 min
Hold at 240° C. for 8 min

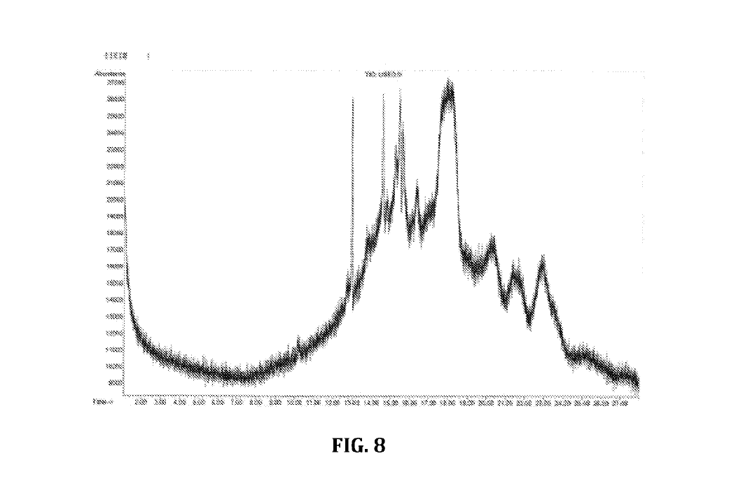

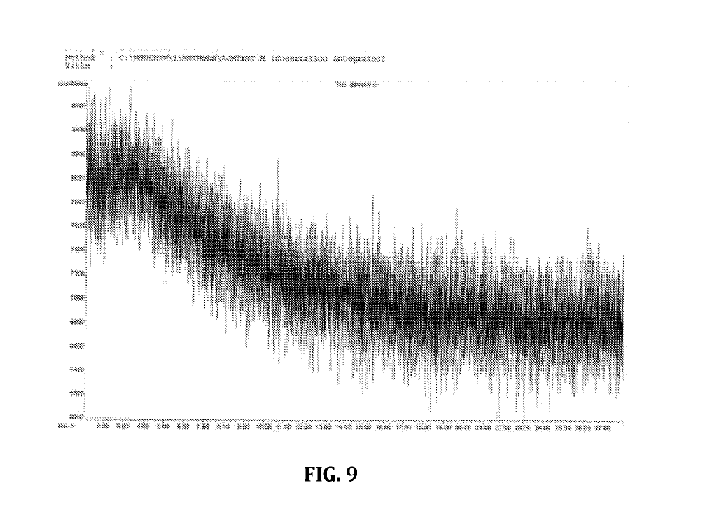

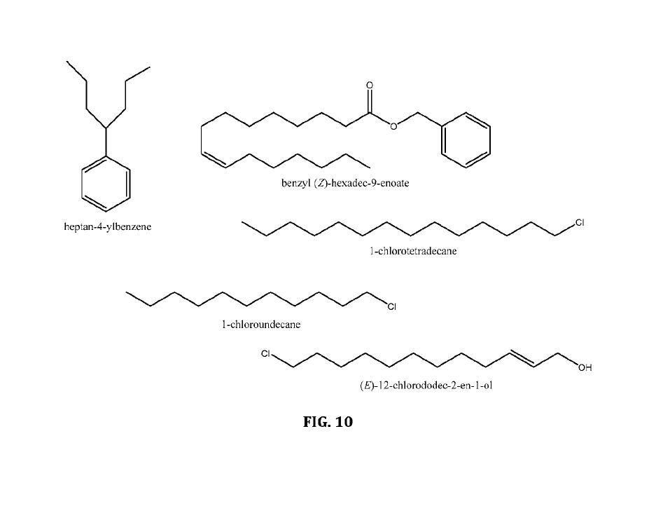

As shown in FIG. 8, analysis of the urine containing sample provided an array of aromatic compounds responsible for the characteristic smell. Then, one pump of the spray was dispensed inside the bag and allowed to react for 5 min. FIG. 9: Gas chromatogram obtained with the headspace of used cat litter after treatment with the spray containing liposomes. Further analysis of the fragments corresponding to the most abundant components released by the used litter were investigated with the assistance of a database. The most probable targets are included in FIG. 10.

1. A composition comprising a lipid/fatty amino acid derivative liposome encapsulating an oxidant solution.

2. The composition of claim 1, wherein the fatty amino acid is an alanine, serine, or cysteine derivative.

3. The composition of claim 1, wherein the oxidant is ozone or peroxide.

4. The composition of claim 1, wherein the composition is formulated to be sprayed.

5. The composition of claim 1, wherein the composition is formulated as a liquid, gel, lotion or cream.

6. An aerosol container comprising a composition of claim 1.

7. A methods for reducing the scent of a subject and/or object comprising contacting the subject and/or object with an effective amount of a liposome composition of claim 1.

8. The method of claim 7, wherein the composition is sprayed, rubbed, and/or dispersed on the clothes, skin, or surface of an individual and/or object.
